SEQUENCE LISTING (2 sequences)

SEQ ID NO: 1 - DNA, 23 bp, synthetic construct
  source 1..23: mol_type = other DNA
  modified_base 1: mod_base = OTHER; note = 2'-O-methyladenosine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methylguanosine-3'-phosphate
  modified_base 4: mod_base = OTHER; note = 2'-O-methyladenosine-3'-phosphate
  modified_base 6: mod_base = OTHER; note = 2'-O-methyluridine-3'-phosphate
  modified_base 7: mod_base = OTHER; note = guanosine-2'-phosphate
  modified_base 8: mod_base = OTHER; note = 2'-O-methylcytidine-3'-phosphate
  modified_base 9: mod_base = OTHER; note = 2'-O-methyluridine-3'-phosphate
  modified_base 10: mod_base = OTHER; note = 2'-O-methylguanosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methyladenosine-3'-phosphate
  modified_base 16: mod_base = OTHER; note = 2'-deoxy-2'-fluorouridine-3'-phosphate
  modified_base 17: mod_base = OTHER; note = 2'-O-methyladenosine-3'-phosphate
  modified_base 18: mod_base = OTHER; note = 2'-O-methylcytidine-3'-phosphate
  modified_base 19: mod_base = OTHER; note = 2'-O-methyluridine-3'-phosphate
  modified_base 20: mod_base = OTHER; note = 2'-O-methylcytidine-3'-phosphate
  modified_base 21: mod_base = OTHER; note = 2'-O-methylcytidine-3'-phosphorothioate
  modified_base 22: mod_base = OTHER; note = 2'-O-methylcytidine-3'-phosphorothioate
  modified_base 23: mod_base = OTHER; note = 2'-O-methylcytidine
  misc_feature 1..4: note = RNA
  misc_feature 6..23: note = RNA
  misc_feature 5: note = DNA
atgaatgctgagaaatactcccc

SEQ ID NO: 2 - DNA, 23 bp, synthetic construct
  source 1..23: mol_type = other DNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroadenosine-3'-phosphorothioate
  modified_base 3: mod_base = OTHER; note = 2'-O-methyluridine-3'-phosphate
  modified_base 4: mod_base = OTHER; note = 2'-O-methylcytidine-3'-phosphate
  modified_base 6: mod_base = OTHER; note = 2'-O-methylguanosine-3'-phosphate
  modified_base 7: mod_base = OTHER; note = cytidine-2'-phosphate
  modified_base 8: mod_base = OTHER; note = 2'-O-methyluridine-3'-phosphate
  modified_base 9: mod_base = OTHER; note = 2'-O-methyluridine-3'-phosphate
  modified_base 10: mod_base = OTHER; note = 2'-O-methyluridine-3'-phosphate
  modified_base 11: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-deoxy-2'-fluoroadenosine-3'-phosphate
  modified_base 15: mod_base = OTHER; note = 2'-O-methylguanosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methylcytidine-3'-phosphate
  modified_base 20: mod_base = OTHER; note = 2'-O-methylguanosine-3'-phosphate
  modified_base 21: mod_base = OTHER; note = 2'-O-methylcytidine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
  misc_feature 1..4: note = RNA
  misc_feature 6..11: note = RNA
  misc_feature 13..15: note = RNA
  misc_feature 17..23: note = RNA
  misc_feature 5: note = DNA
  misc_feature 12: note = DNA
  misc_feature 16: note = DNA
tatcagcttttccagggtcgccg

(19) United States
(12) Patent Application Publication
(10) Pub. No.: US 2024/0317794 A1
(43) Pub. Date: Sep. 26, 2024

(54) 3'-OH RNA-CONTAINING OLIGONUCLEOTIDE DEPROTECTION

(71) Applicant: ALNYLAM PHARMACEUTICALS, INC., Cambridge, MA (US)

(72) Inventors: Anna Balanov, Cambridge, MA (US); Gary Shen, Cambridge, MA (US); Fred Briones, Cambridge, MA (US); Ligang Zhang, Cambridge, MA (US); Lubomir Nechev, Cambridge, MA (US)

(21) Appl. No.: 18/613,537

(22) Filed: Mar. 22, 2024

Related U.S. Application Data
(60) Provisional application No. 63/453,832, filed on Mar. 22, 2023.

(51) Int. Cl. C07H 21/04; C12N 15/10

(52) U.S. Cl. CPC C07H 21/04; C12N 15/101

(57) ABSTRACT

The disclosure relates to a method of deprotecting a 3′-hydroxyl of an oligonucleotide having the 3′-hydroxyl protected by a silyl protecting group, comprising contacting the oligonucleotide with a mixture comprising DMSO and a deprotecting reagent. The disclosure also relates to a method of recovering a synthesized oligonucleotide from a solid support based on the method of deprotecting a 3′-hydroxyl of an oligonucleotide. The disclosure also relates to a 3′-hydroxyl protected oligonucleotide intermediate involved during the method of deprotecting the oligonucleotide.

### CROSS-REFERENCE TO RELATED APPLICATIONS

This application claims benefit of priority to U.S. Provisional Application No. 63/453,832, filed Mar. 22, 2023, which is herein incorporated by reference in its entirety.

### SEQUENCE LISTING

The instant application contains a Sequence Listing which has been submitted in XML format and is hereby incorporated by reference in its entirety. Said XML copy, created on Mar. 21, 2024, is named 29520.1514-US__ALN-502-US__SL.xml and is 30,243 bytes in size.

### FIELD OF THE INVENTION

This invention generally relates to the field of chemical synthesis of oligonucleotides, in particular oligonucleotides comprising at least one 3′-OH RNA modification.

### BACKGROUND

Modified oligonucleotides are of great value in molecular biological research and in therapeutic applications. The ease and yield of chemical synthesis of many modified oligonucleotides are still low to this date, partially because commonly used protecting groups are unstable under many of the conditions employed for deprotecting chemically synthesized oligonucleotides. These issues are especially problematic when preparing oligonucleotides comprising at least one nucleotide having a 3′-hydroxyl group.

An important component of oligonucleotide synthesis is the installation and removal of protecting groups. The 5′-hydroxyl group and the 2′-position typically need to be protected during oligonucleotide synthesis, and for a 3′-OH RNA containing oligonucleotide, 3′-position also needs to be protected during oligonucleotide synthesis. Each of these positions' protecting groups needs to be removed at different times, which has led to more complex synthesis and incomplete and inefficient deprotection. Incomplete installation or removal of a protecting group lowers the overall yield of the synthesis and introduces impurities that are often difficult to remove from the final product.

Thus, there remains a need in the art for an improved process for manufacturing an oligonucleotide comprising at least one nucleotide having a 3′-hydroxyl group, including a better deprotecting reagent and conditions for deprotecting a 3′-hydroxyl containing oligonucleotide, and improved purification procedures. This invention answers that need.

### SUMMARY OF THE INVENTION

This disclosure relates to a manufacturing process for a 3′-hydroxyl (3′-OH RNA)-containing 2′-5′-oligonucleotide, including synthesis, deprotection, and purification, and a 3′-hydroxyl protected oligonucleotide intermediate involved during this manufacturing process.

One aspect of the invention relates to a method of deprotecting a 3′-hydroxyl of an oligonucleotide having the 3′-hydroxyl protected by a silyl protecting group, comprising contacting the oligonucleotide with a mixture comprising DMSO and a deprotecting reagent having a formula of (R)₄N⁺F⁻, wherein each R is independently alkyl, aryl, heteroaryl, aralkyl, or heteroaralkyl, under conditions sufficient for the deprotecting reagent to remove the silyl protecting group.

Another aspect of the invention provides a 3′-hydroxyl protected oligonucleotide intermediate, involved during manufacture of the oligonucleotides comprising at least one nucleotide having a 3′-hydroxyl group. The oligonucleotide intermediate has the structure of formula (I):

A-[C]ₙ-E (I), or a salt thereof (e.g., a sodium salt),

wherein:
A is

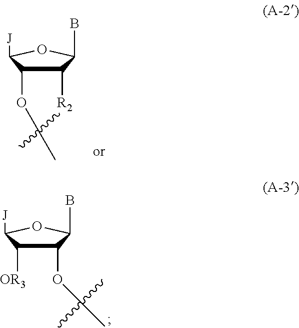

C is

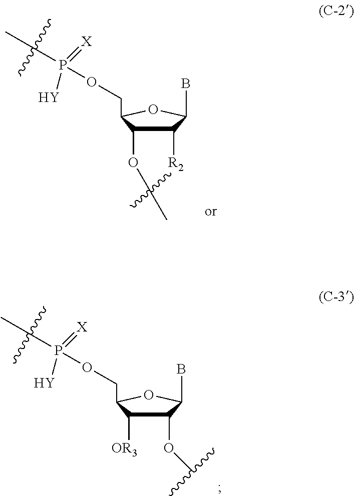

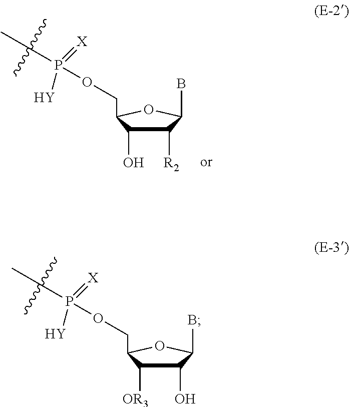

E is
provided that the oligonucleotide intermediate contains at least one (A-3′), (C-3′), or (E-3′);
n is 1-50;
B is a modified or unmodified nucleobase;
J is

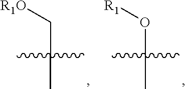

or a phosphorous-containing group or moiety;
R₁ is H, a hydroxyl protecting group, or a phosphorous-containing group or moiety;
R₂ is H, halo, OR₅, or NR₆R₇;
R₃ is —(CH₂)ₘSi(R₄)₃;
each R₄ is independently optionally substituted alkyl, aryl, aralkyl, alkaryl, cycloalkyl, alkenyl, cycloalkenyl, alkynyl or cycloalkynyl;
m is 0, 1, 2, or 3;
R₅, R₆, and R₇ are each independently H or optionally substituted alkyl, alkoxyalkyl, aryl, aralkyl, alkaryl, cycloalkyl, alkenyl, cycloalkenyl, alkynyl or cycloalkynyl; or R₆ and R₇ are linked to form a heterocyclyl; and
X and Y are each independently O or S.

Additional aspects, advantages and features of the invention are set forth in this specification, and in part will become apparent to those skilled in the art on examination of the following, or may be learned by practice of the invention. The inventions disclosed in this application are not limited to any particular set of or combination of aspects, advantages and features. It is contemplated that various combinations of the stated aspects, advantages and features make up the inventions disclosed in this application.

### BRIEF DESCRIPTION OF THE DRAWINGS

FIG. 1 shows the structure of an exemplary oligonucleotide containing a nucleotide having a 3′-hydroxyl group (3′-OH RNA modification). This single-stranded oligonucleotide (A-2114132; SEQ ID NO:1) has chemistries of 2′-H (deoxyribonucleotides), 2′-OMe, and 2′-F modified ribonucleotides. There is one 3′-OH RNA modification in the seventh position from the 5′ end. This strand contains 23 nucleotides and contains four phosphorothioate linkages—two at the 3′ end and two at the 5′ end. This strand is connected through 3′-5′ phosphodiester linkages or 3′-5′ phosphorothioate linkages except one 2′-5′ phosphodiester linkage, thus forming the sugar-phosphate backbone of the oligonucleotide.

FIG. 2A shows the AX-HPLC of the oligonucleotide intermediate, before the desilylation step, showing the traces for the TIPS-protected oligonucleotide (A-2114132).

FIG. 2B shows the IPRP-HPLC of the oligonucleotide intermediate, before the desilylation step, showing the traces for the TIPS-protected oligonucleotide (A-2114132).

FIG. 3 shows the results of monitoring the removal of the TIPS protective group of A-2114132 by AX-HPLC.

FIG. 4A shows the AX-HPLC trace of the desalted crude oligonucleotide (A-2114132), before the purification step. FIG. 4B shows the IPRP-HPLC trace of the desalted crude oligonucleotide (A-2114132), before the purification step.

FIG. 5 shows the structure of an exemplary oligonucleotide containing a nucleotide having a 3′-hydroxyl group (3′-OH RNA modification). This single-stranded oligonucleotide (A-2403673; SEQ ID NO:2) has chemistries of 2′-H (deoxyribonucleotides), 2′-OMe, 2′-F modified, and 5′-VP modified ribonucleotides. There is one 3′-OH RNA modification in the seventh position from the 5′ end. This strand contains 23 nucleotides and contains four phosphorothioate linkages—two at the 3′ end and two at the 5′ end. This strand is connected through 3′-5′ phosphodiester linkages or 3′-5′ phosphorothioate linkages except one 2′-5′ phosphodiester linkage, thus forming the sugar-phosphate backbone of the oligonucleotide.

### DETAILED DESCRIPTION OF THE INVENTION

This disclosure provides a description of a manufacturing process of a single-stranded oligonucleotide comprising at least one nucleotide having a 3′-hydroxyl group, the process including synthesis, cleavage and deprotection, desilylation, crude ultrafiltration, and purification.

Embodiments of this disclosure provide an improved method for manufacturing an 2′-5′ oligonucleotide comprising at least one nucleotide having a 3′-hydroxyl group, that is, a 3′-RNA (3′-hydroxyl and 2′-5′ phosphodiester linkage) modification. A protected phosphoramidite comprising a silyl protected 3′-hydroxyl group (e.g., trialkylsilyl such as triisopropylsilyl) is coupled to a free hydroxyl (e.g., 5′-OH, 3′-OH, or 2′-OH) on a nucleoside or an oligonucleotide. In one embodiment, a protected phosphoramidite comprising a silyl protected (e.g., trialkylsilyl such as triisopropylsilyl) 3′-hydroxyl group is coupled to a 5′-OH on a nucleoside or an oligonucleotide.

After synthesis, certain embodiments of the invention relates to a method of recovering a synthesized oligonucleotide from a solid support, comprising:
i) contacting a synthesized oligonucleotide bound to a solid support with a base treatment to cleave the synthesized oligonucleotide from the solid support and to remove amino protecting groups from the synthesized oligonucleotide; and
ii) contacting the oligonucleotide with a mixture comprising DMSO and a deprotecting reagent having a formula of (R)₄N⁺F⁻, wherein each R is independently alkyl, aryl, heteroaryl, aralkyl, or heteroaralkyl, under conditions sufficient for the deprotecting reagent to remove the silyl protecting group.

Another aspect of the invention relates to a method of deprotecting a 3′-hydroxyl of an oligonucleotide having the 3′-hydroxyl protected by a silyl protecting group, comprising contacting the oligonucleotide with a mixture comprising DMSO and a deprotecting reagent having a formula of (R)₄N⁺F⁻, wherein each R is independently alkyl, aryl, heteroaryl, aralkyl, or heteroaralkyl, under conditions sufficient for the deprotecting reagent to remove the silyl protecting group.

### Solid Phase Oligonucleotide Synthesis

The manufacture process starts with oligonucleotide synthesis. Typically, solid phase synthesis is employed for oligonucleotide synthesis. Solid-phrase oligonucleotide synthesis is known to one skilled in the art. Suitable solid phase techniques, including automated synthesis techniques, are described in F. Eckstein (ed.), Oligonucleotides and Analogues, a Practical Approach (Oxford University Press, New York 1991), which is incorporated herein by reference in its entirety. Methods and reagents for coupling nucleoside phosphoramidite monomers to 2′-hydroxyl groups are also known in the art. Thus, the oligonucleotide can be prepared using procedures and equipment known to those skilled in the art. The assembly of the nucleotide intermediate for 2′-hydroxyl nucleotide is from the 3′ to the 5′ terminus while the assembly of the nucleotide intermediate for 3′-hydroxyl nucleotide is from 2′ to 5′ terminus. An exemplary process for synthesizing oligonucleotide containing a nucleotide having a 3′-hydroxyl group (3′-RNA modification) is illustrated in Example 1, Section 1.3.1.

### Deprotections

After synthesis is complete, the oligonucleotide can be deprotected, e.g., using methods and reagents to remove any protecting groups on the oligonucleotide to obtain the desired product.

Accordingly, in some embodiments, the method further relates to recovering a synthesized oligonucleotide from a solid support or a method of deprotecting an oligonucleotide having a 3′-hydroxyl protected by a silyl protecting group.

The method may optionally comprise step i) contacting a synthesized, nucleobase-protected oligonucleotide having a 3′-hydroxyl protected by a silyl protecting group bound to a solid support with a base under conditions suitable to cleave the oligonucleotide from the solid support and to remove amino protecting groups from the nucleobase-protected oligonucleotide, to provide the oligonucleotide having the 3′-hydroxyl protected by a silyl protecting group.

The method comprises step ii) contacting the oligonucleotide with a mixture comprising DMSO and a deprotecting reagent having a formula of (R)₄N⁺F⁻, wherein each R is independently alkyl, aryl, heteroaryl, aralkyl, or heteroaralkyl, under conditions sufficient for the deprotecting reagent to remove the silyl protecting group.

Step i) involves treating the synthesized oligonucleotide to remove any protecting groups on the oligonucleotide not related to the 3′-hydroxyl protection.

This step cleaves the synthesized oligonucleotide from the solid support and removes amino protecting groups (e.g., nucleobase protecting groups) from the synthesized oligonucleotide. Thus, the removal of the nucleobase protecting groups occurs concomitantly with the cleavage of the oligonucleotide from the solid support. Thus, in some embodiments, the method comprises, prior to removing the silyl protecting group: contacting a nucleobase-protected oligonucleotide having a 3′-hydroxyl protected by a silyl protecting group bound to a solid support with a base under conditions suitable to cleave the oligonucleotide from the solid support and to remove amino protecting groups from the nucleobase-protected oligonucleotide, to provide the oligonucleotide having the 3′-hydroxyl protected by a silyl protecting group.

Exemplary bases for deprotection in oligonucleotide synthesis, prior to removing the 3′-hydroxyl protective groups, include, but are not limited to, ammonium hydroxide, methylamine, and mixtures thereof. In some embodiments, the base is ammonia or ammonia in a solvent (such as water, ethanol, or DMSO).

The base treatment can suitably be carried out at room temperature or elevated temperature. “Room temperature” includes ambient temperatures from about 20° C. to about 30° C. “Elevated temperature” includes temperatures higher than 30° C. For example, elevated temperature can be a temperature between about 32° C. to about 65° C. In one embodiment, the base treatment is at about 33 to about 37° C., for instance, at about 35° C. The duration for the base treatment is often in the order of minutes (such as, for example 5, 10, 15, 20, 25, 30, 45 or 60 minutes) to hours (such as, for example, 2 hours, 3 hours, 4 hours, 5 hours, 10 hours, 15 hours, 24 hours or longer). In some embodiments, the base treatment is at about 35° C. for about 12-22 hours, or for about 16-20 hours.

In some embodiments, the amino protecting groups are exocyclic amino (nucleobase) protecting groups, and the base is ammonia or a mixture of ammonia and a solvent (e.g., water, ethanol, or DMSO). The base treatment may be carried out at about 33-37° C. for about 16-20 hours. The concentration of ammonia may range from about 5% to about 50% (such as, for example, about 5%, about 10%, about 15%, about 20%, about 25%, about 30%, about 35%, about 40%, about 45%, or about 50%). When a mixture of ammonia and a solvent is used, the volume ratio of ammonia and the solvent (e.g., water, ethanol, or DMSO) may be up to about 10:1 v/v ratio, for instance, up to 9:1 v/v ratio, 8:1 v/v ratio, 7:1 v/v ratio, 6:1 v/v ratio, 5:1 v/v ratio, 4:1 v/v ratio, or 3:1 v/v ratio. In one embodiments, the base is a mixture of about 30% ammonia and a solvent (e.g., water, ethanol, or DMSO) at ammonia to solvent ratio of up to 3:1 v/v (e.g., about 3:1 v/v, about 2.5:1 v/v, about 2:1 v/v, about 1.5:1, or about 1:1 v/v). In one embodiments, the base is about 30% ammonia. In one embodiment, the base treatment is carried out at about 33 to about 37° C. (e.g., 35° C.) for about 16-20 hours.

The method may also involve a 5′-hydroxyl deprotection prior to step i). In some embodiments, the method further comprises, prior to step i), an acid treatment step, to remove a 5′-hydroxyl protecting group of the synthesized oligonucleotide. The 5′-hydroxyl protecting group may be selected from the group consisting of 4,4′-dimethoxytrityl (DMT), monomethoxytrityl (MMT), 9-fluorenylmethylcarbonate (Fmoc), o-nitrophenylcarbonyl, p-phenylazophenylcarbonyl, phenylcarbonyl, p-chlorophenylcarbonyl, and 5′-(α-methyl-2-nitropiperonyl)oxycarbonyl (MeNPOC).

The acid is a mild acid that does not remove the 3′-hydroxyl protective group. Suitable acids include but are not limited to acetic acid, dichloroacetic acid (DCA), trichloroacetic acid (TCA), or trifluoroacetic acid (TFA). In some embodiments, the acid is dichloroacetic acid (DCA). In one embodiment, the 5′-hydroxyl protecting group of the nucleotide/oligonucleotide is dimethoxytrityl (DMT), and the acid is DCA (e.g., 10% DCA in toluene, or 3% DCA in dichloromethane).

The method may also involve a phosphate deprotection prior to the base treatment of step i) to remove amino protecting groups from the nucleobase-protected oligonucleotide. In some embodiments, the method further comprises, prior to the base treatment of step i), but after the acid treatment step, contacting a fully-protected oligonucleotide (i.e., all necessary protecting groups from solid state synthesis, e.g., a phosphate/phosphorothioate-protected and nucleobase-protected oligonucleotide) having a 3′-hydroxyl protected by a silyl protecting group bound to a solid support with a base (e.g., a weak base) under conditions suitable to remove a phosphate protecting group of the oligonucleotide, to provide the nucleobase-protected oligonucleotide having a 3′-hydroxyl protected by a silyl protecting group.

Suitable weak bases remove the phosphate protecting group, but do not remove amino protecting groups (e.g., nucleobase protecting groups) from the synthesized oligonucleotide; nor do they remove the 3′-hydroxyl protective group from the synthesized oligonucleotide. For instance, the weak bases can be diethyl amine or triethylamine, optionally in an organic solvent. In some embodiments, the phosphate protecting group is cyanoethyl, and the weak base is diethyl amine (DEA), e.g., 20% DEA in acetonitrile. In some embodiments, the phosphate protecting group is cyanoethyl, and the weak base is triethyl amine (TEA), e.g., 1:1 TEA/acetonitrile.

Both the acid treatment to deprotect 5′-hydroxyl and the weak base treatment to deprotect phosphate occur prior to step i). That is to say, both deprotection steps occur before the synthesized oligonucleotide is cleaved from the solid support.

With the treatments described herein, at the end of the base treatment of step i), the 3′-hydroxyl group of the nucleotide is still protected with a silyl protecting group.

Step ii) involves treating the partially deprotected oligonucleotide from step i) to remove the protective group on the 3′-hydroxyl of the oligonucleotide. The 3′-hydroxyl group is typically protected with a silyl protecting group.

Some embodiments of this invention thus also relates to a method of deprotecting a 3′-hydroxyl of an oligonucleotide having the 3′-hydroxyl protected by a silyl protecting group. The method comprises contacting the oligonucleotide with a mixture comprising DMSO and a deprotecting reagent having a formula of (R)₄N⁺F⁻, wherein each R is independently alkyl, aryl, heteroaryl, aralkyl, or heteroaralkyl, under conditions sufficient for the deprotecting reagent to remove the silyl protecting group, and convert the silyl-protected hydroxyl group to a free hydroxyl group at the 3′ position of the nucleotide.

In some embodiments, the silyl protecting group to protect the 3′-hydroxyl of the oligonucleotide is trialkylsilyl or —CH₂Si(alkyl)₃ (e.g., each alkyl may independently be a C₁-C₆ alkyl (e.g., C₁-C₄ alkyl)). In one embodiment, the silyl protecting group to protect the 3′-hydroxyl of the oligonucleotide is triisopropylsilyl (TIPS). In one embodiment, the silyl protecting group to protect the 3′-hydroxyl of the oligonucleotide is tert-butyldimethylsilyl.

A common silyl deprotecting reagent used in the art is HF.pyridine. The inventors of this invention have discovered, however, that (R)₄N—F in DMSO can be used as a milder and more efficient silyl deprotecting reagent for the deprotection of the 3′-hydroxyl of an oligonucleotide. R may be independently alkyl, aryl, heteroaryl, aralkyl, or heteroaralkyl. In some embodiments, each R is independently an alkyl, for instance, a C₁-C₆ alkyl (e.g., C₁-C₄ alkyl).

In some embodiments, the deprotecting reagent is tetraethylammonium fluoride (TEAF). In some embodiments, the deprotecting reagent is tetraethylammonium fluoride hydrate (TEAF⋅xH₂O). In some embodiments, the deprotecting reagent is tetra-n-butylammonium fluoride (TBAF).

In some embodiments, the silyl protecting group to protect the 3′-hydroxyl of the oligonucleotide is triisopropylsilyl (TIPS), and to deprotect the 3′-hydroxyl group, the oligonucleotide is contacted with a mixture comprising a tetraethylammonium fluoride (such as solid TEAF) in aqueous DMSO solution. The concentration of TEAF in the aqueous DMSO used for deprotection may range from about 0.1 M to about 2 M, for instance, from about 0.5 M to about 1.5 M, from about 0.5 M to about 1.1 M, or from about 0.8 M to about 1.1 M (such as, for example, about 0.1 M, about 0.15 M, about 0.2 M, about 0.25 M, about 0.3 M, about 0.35 M, about 0.4 M, 0.45 M, about 0.5 M, about 0.55 M, about 0.6 M, about 0.65 M, about 0.7 M, about 0.75 M, about 0.8 M, about 0.85 M, about 0.9 M, about 0.95 M, about 1.0 M, about 1.05 M, about 1.1 M, about 1.15 M, about 1.2 M, about 1.25 M, about 1.3 M, about 1.35 M, about 1.4 M, about 1.45 M, about 1.5 M, about 1.55 M, about 1.6 M, about 1.65 M, about 1.7 M, about 1.75 M, about 1.8 M, about 1.85 M, about 1.9 M, about 1.95 M, or about 2.0 M). The ratio between the oligonucleotide solution to the deprotecting reagent solution (e.g., TEAF in the aqueous DMSO) may range between about 0.5:1 to about 2:1 v/v, for instance, between about 0.6:1 to about 1.8:1 v/v, between about 0.7:1 to about 1.6:1 v/v, between about 0.8:1 to about 1.4:1 v/v, or between about 0.9:1 to about 1.2:1 v/v (such as, for example, about 0.5:1 v/v, about 0.6:1 v/v, about 0.7:1 v/v, about 0.8:1 v/v, about 0.9:1 v/v, about 1:1 v/v, about 1.1:1 v/v, about 1.2:1 v/v, about 1.3:1 v/v, about 1.4:1 v/v, about 1.5:1 v/v, about 1.6:1 v/v, about 1.7:1 v/v, about 1/8:1 v/v, about 1.9:1 v/v, or about 2:1 v/v). In one embodiment, the partially deprotected oligonucleotide solution, treated after the base treatment of step i), is contacted with a solid TEAF in a DMSO solution (at about 0.5 M to about 1.1 M, or 0.8 M to about 1.1 M TEAF in DMSO, e.g., about 0.9 M TEAF in DMSO), with the ratio of the oligonucleotide solution to the TEAF/DMSO solution of between about 0.5:1 to about 2:1 v/v (e.g., about 0.9:1 to about 1.2:1 v/v, or about 1:1 v/v).

The deprotecting reagent (tetraethylammonium fluoride) may be added all at once, or added in portions for a duration of time, during the desilylation step. For instance, TEAF may be added to the reactor prior to the addition of the oligonucleotide solution and/or DMSO; alternatively, TEAF may be added to the reactor after the addition of the oligonucleotide solution and/or DMSO. The deprotecting reagent (tetraethylammonium fluoride) may be added as different forms. For instance, TEAF may be pre-mixed with DMSO solution and added to the reactor as TEAF/DMSO mixture; DMSO and TEAF may be added separately with TEAF added as solid, in the form of either TEAF or TEAFxH2O. When TEAF is added in portions, at various times, TEAF may be added in different forms. For instance, TEAF may be added as a pre-mixture with DMSO solution first, and may be added separately from DMSO as a solid in a later stage, or vice versa.

The deprotecting step for removing a silyl protective group (e.g., trialkylsilyl such as triisopropylsilyl) from the protected 3′-hydroxyl of the oligonucleotide can suitably be carried out at room temperature or elevated temperature. For example, the desilylation step can be carried out at about 50° C. or higher, about 55° C. or higher, about 60° C. or higher, or about 65° C. or higher. In some embodiments, the desilylation step (contacting) is carried out at a temperature ranging from about 60° C. to about 65° C., e.g., at around 60° C. or around 65° C. The duration for the desilylation step is often in the order of minutes (such as, for example 5, 10, 15, 20, 25, 30, 45 or 60 minutes) to hours (such as, for example, 2 hours, 3 hours, 4 hours, 5 hours, or 10 hours). In some embodiments, the desilylation step (contacting) is carried out for about 30 minutes or longer, about 1 hour or longer, or about 2 hours or longer. In some embodiments, the desilylation step (contacting) is carried out for about 2 hours.

An exemplary process for deprotecting oligonucleotide containing a nucleotide having a 3′-hydroxyl group (3′-OH RNA modification) is illustrated in Example 1, Section 1.3.2.

After deprotection, the desired oligonucleotide product can be isolated and purified using methods known in the art. Such methods include, but are not limited to, filtration and/or one or more chromatographic purifications.

In some embodiments, the manufacturing method further comprises filtration to desalt the oligonucleotide, e.g., ultrafiltration.

In some embodiments, the manufacturing method further comprises one or more chromatographic purifications to purify the oligonucleotide. Any chromatographic purification methods for oligonucleotide purification known to one skilled in the art may be used herein.

### 3′-Hydroxyl Protected Oligonucleotide Intermediate

Another aspect of the invention provides a 3′-hydroxyl protected oligonucleotide intermediate, involved during manufacture of the oligonucleotides comprising at least one nucleotide having a 3′-hydroxyl group. The oligonucleotide intermediate has the structure of formula (I): A-[C]ₙ-E (I), or a salt thereof (e.g., a sodium salt),

wherein:
A is

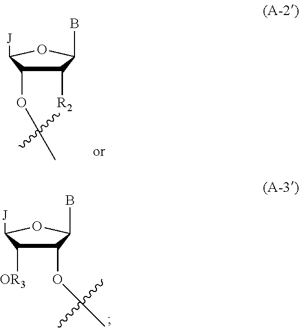

C is

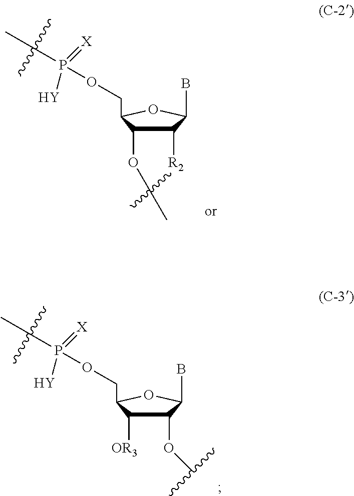

E is

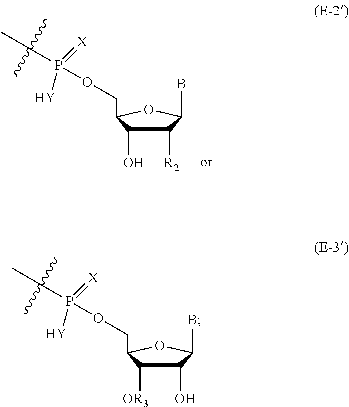

provided that the oligonucleotide intermediate contains at least one (A-3′), (C-3′), or (E-3′);
n is 1-50;
B is a modified or unmodified nucleobase;
J is

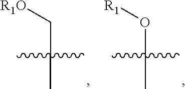

or a phosphorous-containing group or moiety;
R₁ is H, a hydroxyl protecting group, or a phosphorous-containing group or moiety;
R₂ is H, halo, OR₅, or NR₆R₇;
R₃ is —(CH₂)ₘSi(R₄)₃;
each R₄ is independently optionally substituted alkyl, aryl, aralkyl, alkaryl, cycloalkyl, alkenyl, cycloalkenyl, alkynyl or cycloalkynyl;
m is 0, 1, 2, or 3;
R₅, R₆, and R₇ are each independently H or optionally substituted alkyl, alkoxyalkyl, aryl, aralkyl, alkaryl, cycloalkyl, alkenyl, cycloalkenyl, alkynyl or cycloalkynyl; or R₆ and R₇ are linked to form a heterocyclyl; and
X and Y are each independently O or S.

In the above formulas, A is a nucleotide at the 5′ terminus, and can be represented by A-2′ or A-3′; C is an internal nucleotide, and can be represented by C-2′ or C-3′; and E is a nucleotide at the 3′ terminus, and can be represented by E-2′ or E-3′ A-3′, C-3′, and E-3′ each represent a nucleotide that has 3′-hydroxyl protected by R₃, a silyl protecting group, —Si(R₄)₃. The oligonucleotide intermediate contains at least one A-3′, C-3′, or E-3′, i.e., the oligonucleotide intermediate contains at least one nucleotide having the 3′-hydroxyl protected by a silyl protecting group, —Si(R₄)₃.

In some embodiments, A is A-3′.

In some embodiments, at least one C is C-3′.

In some embodiments, E is E-3′.

R₃ is —(CH₂)ₘSi(R₄)₃. The integer m is 0, 1, 2, or 3. In some embodiments, m is 0 or 1. In some embodiments, R₃ is —Si(R₄)₃ or —CH₂Si(R₄)₃. Each R₄ is independently alkyl, aryl, aralkyl, alkaryl, cycloalkyl, alkenyl, cycloalkenyl, alkynyl or cycloalkynyl, optionally substituted with one or more substituents. For example, each R⁴ can be independently an optionally substituted C₁-C₆ alkyl. Exemplary alkyls for R⁴ include, but are not limited to methyl, ethyl, propyl, isopropyl, butyl, 2-methylpropuyl, t-butyl, and pentyl. In some embodiments, each R⁴ is isopropyl. In some embodiments, one R⁴ is tert-butyl, and the other two R⁴ are methyl. In some embodiments, m is 0 or 1, and each R⁴ is isopropyl. In some embodiments, m is 0 or 1; and one R⁴ is tert-butyl, and the other two R⁴ are methyl.

The length of the oligonucleotide can range from 3-60 nucleotides, 5-40 nucleotides, for instance, 35, 30, 25, 24, 23, 22, 21, 20, 19, 18, 17, 16, or 15 nucleotides. The integer n is the total length minus the two terminal nucleotides, for instance, n can range from 1-58, 3-38, or 13-33, for instance, 35, 34, 33, 32, 31, 30, 29, 28, 27, 26, 25, 24, 23, 22, 21, 20, 19, 18, 17, 16, 15, 14, or 13. In some embodiments, n is 16-23.

B is a modified or unmodified nucleobase. Optionally, the nucleobase can comprise one or more protecting groups. Exemplary nucleobases include, but are not limited to, adenine, guanine, cytosine, uracil, thymine, inosine, xanthine, hypoxanthine, nubularine, isoguanisine, tubercidine, and substituted or modified analogs of adenine, guanine, cytosine and uracil. For instance, those substituted or modified analogs can be 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and 0-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine,7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N4-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808; Concise Encyclopedia of Polymer Science and Engineering, pages 858-859 (Kroschwitz, J. I., ed. John Wiley & Sons, 1990), and Englisch et al., Angewandte Chemie, International Edition, 30:613 (1991), which are incorporated herein by reference in their entireties.

In some embodiments, the nucleobase can be adenine, guanine, cytosine, uracil, thymine, inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine,2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil,5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil,4-(thio)pseudouracil,2,4-(dithio)psuedouracil,5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, 06-substituted purines, substituted 1,2,4-triazoles, or any O-alkylated or N-alkylated derivatives thereof. In some embodiments, the nucleobase is adenine, guanine, cytosine, or uracil.

R₂ is H, halo, OR₅, or NR₆R₇. R₅, R₆, and R₇ are each independently H or alkyl, alkoxyalkyl, aryl, aralkyl, alkaryl, cycloalkyl, alkenyl, cycloalkenyl, alkynyl or cycloalkynyl, optionally substituted with one or more sustituents; or R₆ and R₇ are linked to form a heterocyclyl, which can be optionally substituted with one or more sustituents. In some embodiment, R₅ is H. In some embodiments, R₅ is alkyl, alkoxyalkyl, aryl, aralkyl, alkaryl, cycloalkyl, alkenyl, cycloalkenyl, alkynyl or cycloalkynyl, optionally substituted with one or more substituents. In some embodiments, R₅ is alkyl (e.g., methyl, ethyl, or propyl) or alkoxyalkyl (e.g., methoxymethyl, methoxyethyl, ethoxymethyl, or ethoxyethyl). In some embodiment, R⁶ and R⁷ are each independently an optionally substituted C₁-C₆ alkyl (e.g., methyl, ethyl, propyl, isopropyl, butyl, 2-methylpropuyl, t-butyl, or pentyl). In some embodiments, R₂ is H, F, OCH₃, or OCH₂CH₂OCH₃.

J is

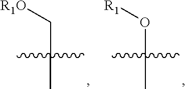

or a phosphorous-containing group or moiety. R₁ is H, a hydroxyl protecting group, or a phosphorous-containing group or moiety.

Any hydroxyl protecting group known and used in the art for hydroxyl protection during oligonucleotide synthesis can be used. Exemplary hydroxyl protecting groups include, but are not limited to, acetyl, benzyl, benzoyl, 2,6-dichlorobenzyl, t-butyldimethylsilyl, t-butyldiphenylsilyl, mesylate, tosylate, dimethoxytrityl such as 4,4′-dimethoxytrityl (DMT), monomethoxytrityl (“MMT”), 9-phenylxanthine-9-yl (Pixyl), 9-fluorenylmethylcarbonate (“Fmoc”), o-nitrophenylcarbonyl, p-phenylazophenylcarbonyl, phenylcarbonyl, p-chlorophenylcarbonyl, and 5′-(α-methyl-2-nitropiperonyl)oxycarbonyl (“MeNPOC”), and 9-(p-methoxyphenyl)xanthine-9-yl (MOX). In one embodiment, R is DMT.

Exemplary phosphorous-containing groups or moieties for J or R₁ include, but are not limited to, 5′-phosphate ((HO)₂(O)P—O-5′), 5′-monophosphate ((HO)₂(O)P—O-5′), 5′-diphosphate ((HO)₂(O)P—O—P(HO)(O)—O-5′), 5′-triphosphate ((HO)₂(O)P—O—(HO)(O)P—O—P(HO)(O)—O-5′), 5′-monothiophosphate (or phosphorothioate) (HO)₂(S)P—O-5′), 5′-monodithiophosphate (or phosphorodithioate) (HO)(HS)(S)P—O-5′), 5′-phosphorothiolate ((HO)₂(O)P—S-5′), 5′-alpha-thiotriphosphate, 5′-beta-thiotriphosphate, 5′-gamma-thiotriphosphate, and 5′-phosphoramidates ((HO)₂(O)P—NH-5′ or (HO)(NH₂)(O)P—O-5′).

In some embodiments, J is

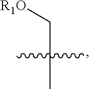

### and R₁ is H.

In some embodiments, J is

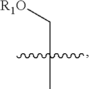

and R₁ is a hydroxyl protecting group.

In some embodiments, J is a 5′-terminus phosphorous-containing group or moiety. For instance, J may be 5′-phosphate

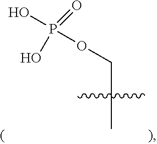 [SMILES: *COP(=O)(O)O]

5′-phosphorothioate

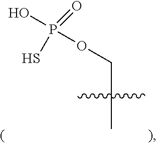 [SMILES: *COP(=O)(O)S]

5′-phosphorodithioate

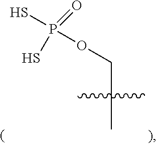 [SMILES: *COP(=O)(S)S]

5′-vinylphosphonate

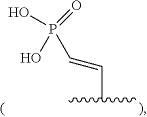 [SMILES: */C=C/P(=O)(O)O]

5′-methylphosphonate

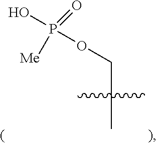 [SMILES: *COP(C)(=O)O]

5′-methoxypropylphosphonate

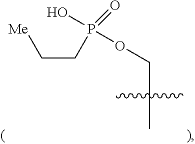 [SMILES: *COP(=O)(O)CCC]

5′-deoxy-5′-C-malonyl

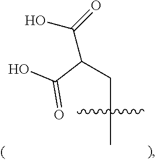 [SMILES: *CC(C(=O)O)C(=O)O]

or a salt thereof (e.g., sodium salt). When J is 5′-vinylphosphonate (5′-VP), the 5′-VP can be either 5′-E-VP isomer (i.e., trans-vinylphosphate,

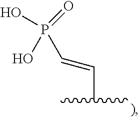 [SMILES: */C=C/P(=O)(O)O]

5′-Z-VP isomer (i.e., cis-vinylphosphate,

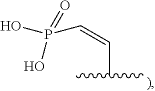 [SMILES: */C=C\P(=O)(O)O]

or mixtures thereof.

The 5′-terminus phosphorus-containing moiety can also include a 5′-phosphate prodrug or 5′-phosphonate prodrug.

In some embodiments, J is

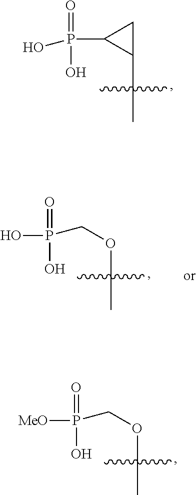 [SMILES: *C1CC1P(=O)(O)O ; *OCP(=O)(O)O ; *OCP(=O)(O)OC]

or a salt (e.g., sodium salt) thereof. In some embodiments, A may be

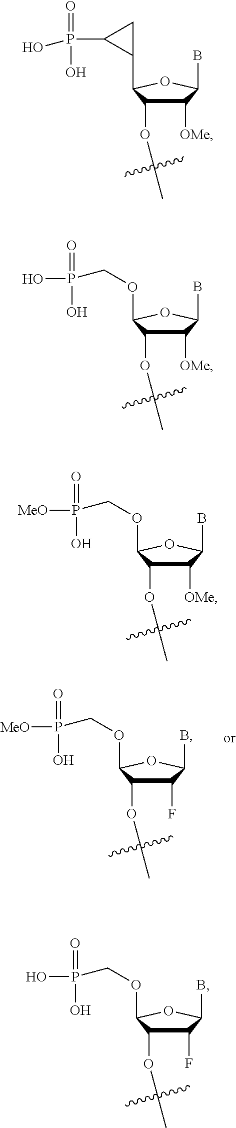

or a salt (e.g., sodium salt) thereof, wherein B is a modified or unmodified nucleobase.

In some embodiments, J is a 5′-phosphate prodrug or 5′-phosphonate prodrug having a structure disclosed in WO2022/147214, which is incorporated herein by reference. For instance, J can be any compounds comprising a structure of formula (I) as disclosed in WO2022/147214. In some embodiments, J may be represented by
Pmmds

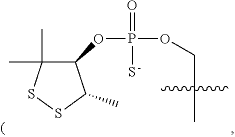 [SMILES: *COP(=O)(S)O[C@H]1[C@H](C)SSC1(C)C]

((4SR,5SR)-3,3,5-trimethyl-1,2-dithiolan-4-ol) phosphodiester);
cPmmds

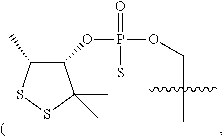 [SMILES: *COP(=O)(S)O[C@H]1[C@@H](C)SSC1(C)C]

((4SR,5RS)-3,3,5-trimethyl-1,2-dithiolan-4-ol) phosphodiester (Cis Pmmds));
PdArls

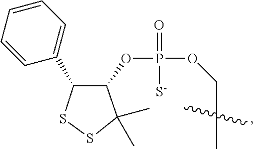 [SMILES: *COP(=O)([S-])O[C@H]1[C@@H](c2ccccc2)SSC1(C)C]

((4SR,5RS)-5-phenyl-3,3-dimethyl-1,2-dithiolan-4-ol) phosphodiester);
PdAr3s

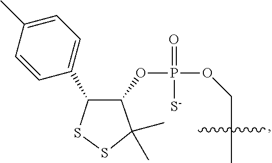 [SMILES: *COP(=O)([S-])O[C@H]1[C@@H](c2ccc(C)cc2)SSC1(C)C]

((4SR,5RS)-5-(4-methylphenyl)-3,3-dimethyl-1,2-dithiolan-4-ol) phosphodiester);
PdAr5s

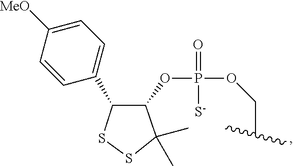 [SMILES: *COP(=O)([S-])O[C@H]1[C@@H](c2ccc(OC)cc2)SSC1(C)C]

((4SR,5RS)-5-(4-methoxyphenyl)-3,3-dimethyl-1,2-dithiolan-4-ol) phosphodiester);

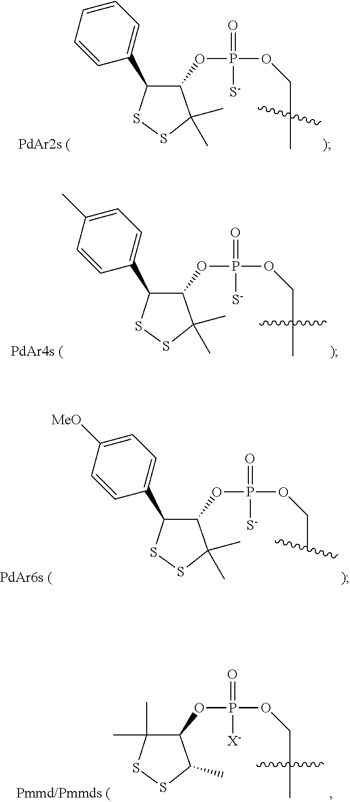

X is O or S);

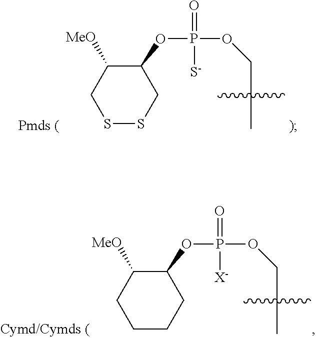

X is O or S); Ptmd/Ptmds

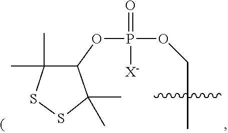

X is O or S), Pd/Pds

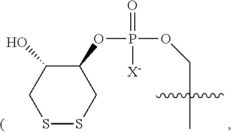

X is O or S).

In some embodiments, in the 3′-hydroxyl protected oligonucleotide intermediate of formula (I) or a salt thereof, A is A-3′, each C is independently C-2′ or C-3′, and E is E-2′ or E-3′; R₃ is —Si(R₄)₃ or —CH₂Si(R₄)₃, and each R⁴ is isopropyl; R₂ is H, F, OCH₃, or OCH₂CH₂OCH₃; each B is independently adenine, guanine, cytosine, or uracil; and X and Y are each independently O or S.

In some embodiments, in the 3′-hydroxyl protected oligonucleotide intermediate of formula (I) or a salt thereof, A is A-2′ or A-3′, at least one C is C-3′ and the remaining Cs are each independently C-2′ or C-3′, and E is E-2′ or E-3′; R₃ is —Si(R₄)₃ or —CH₂Si(R₄)₃, and each R⁴ is isopropyl; R₂ is H, F, OCH₃, or OCH₂CH₂OCH₃; each B is independently adenine, guanine, cytosine, or uracil; and X and Y are each independently O or S.

In some embodiments, in the 3′-hydroxyl protected oligonucleotide intermediate of formula (I) or a salt thereof, A is A-2′ or A-3′, each C is independently C-2′ or C-3′, and E is E-3′; R₃ is —Si(R₄)₃ or —CH₂Si(R₄)₃, and each R⁴ is isopropyl; R₂ is H, F, OCH₃, or OCH₂CH₂OCH₃; each B is independently adenine, guanine, cytosine, or uracil; and X and Y are each independently O or S.

An exemplary structure of the 3′-hydroxyl protected oligonucleotide intermediate is:

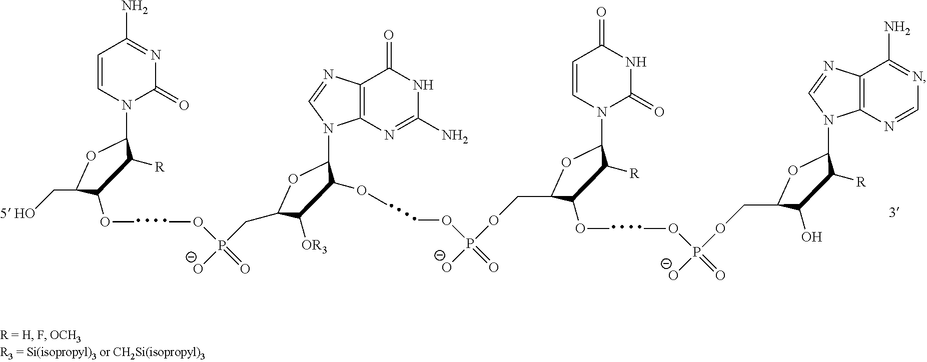

which showing exemplified structures for A-2′, C (C-3′ and C-2′), and E-2′, respectively. The “ . . . ” indicates the omissions of other C units in the oligonucleotide intermediate.

### Definitions

The term “alkyl” refers to the radical of saturated aliphatic groups, including straight or branched-chain alkyl groups, cycloalkyl (alicyclic) groups, alkyl substituted cycloalkyl groups, and cycloalkyl substituted alkyl groups. In some embodiments, a straight or branched chain alkyl has 30 or fewer carbon atoms in its backbone (e.g., C₁-C₃₀ for straight chain, C₃-C₃₀ for branched chain), 20 or fewer carbon atoms in its backbone, or 10 or fewer carbon atoms in its backbone.

The terms “alkenyl” and “alkynyl” refer to unsaturated aliphatic groups analogous in length and possible substitution to the alkyls described above, but that contain at least one double or triple bond, respectively.

Cycloalkyls, cycloalkenyl, or cycloalkynyl have from 3-10 carbon atoms (e.g., 5, 6 or 7 carbon atoms) in their ring structure.

The term “aralkyl” refers to an alkyl group substituted with an aryl group (e.g., an aromatic or heteroaromatic group). For example, a benzyl group (PhCH₂—) is an aralkyl group.

The term “aryl” as used herein includes 5-, 6- and 7-membered single-ring aromatic groups that may include from zero to four heteroatoms, for example, benzene, anthracene, naphthalene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “heteroaryl.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulthydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulthydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulthydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “alkoxyl” or “alkoxy” refers to an alkyl group, as defined above, having an oxygen radical attached thereto. Representative alkoxyl groups include methoxy, ethoxy, propyloxy, tert-butoxy and the like.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. In addition, the substituent can be halogen, azide, alkyl, aralkyl, alkenyl, alkynyl, cycloalkyl, hydroxyl, alkoxyl, amino, nitro, sulthydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, and the like. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. Suitable protecting groups include those disclosed in Greene, T. W.; Wuts, F. G. M. Protective Groups in Organic Synthesis, 2nd ed.; Wiley: New York, 1991, which is incorporated herein by reference in its entirety.

The invention is further illustrated by the following examples, which should not be construed as further limiting. The contents of all references, pending patent applications and published patents, cited throughout this application are hereby expressly incorporated by reference.

### Examples

The invention now being generally described, it will be more readily understood by reference to the following examples which are included merely for purposes of illustration of certain aspects and embodiments of the present invention, and are not intended to limit the invention.

Abbreviations and acronyms used in the examples are shown below.

| Abbreviation | Definition |
|---|---|
| ACN | Acetonitrile |
| CPG | Controlled Pore Glass |
| DEA | Diethyl amine |
| DMF | N,N-dimethylformamide |
| DMSO | Dimethyl sulfoxide |
| DMT | Dimethoxytrityl |
| FLP | Full Length Product |
| HPLC | High Performance Liquid Chromatography |
| IPRP | Ion-Pairing Reverse Phase |
| PADS | Phenylacetyl disulfide |
| TEAF | Tetraethylammonium fluoride |
| TIPS | Triisopropylsilyl |
| v/v | Volume/volume |

### Example 1. Manufacture of an Exemplified 3′-RNA Containing Oligonucleotide

An exemplified oligonucleotide containing a nucleotide having a 3′-hydroxyl group (3′-OH RNA modification) (A-2114132) was manufactured in this example by solid phase synthesis using 3′-O-(2-cyanoethyl) phosphoramidite chemistry, except for G2p which used 2′-0-(2-cyanoethyl) phosphoramidite chemistry followed by chromatographic purification. The synthesis was performed on 2′-OMe A loaded controlled pore glass (CPG) support.

### 1.1 Structure and Sequence of A-2114132

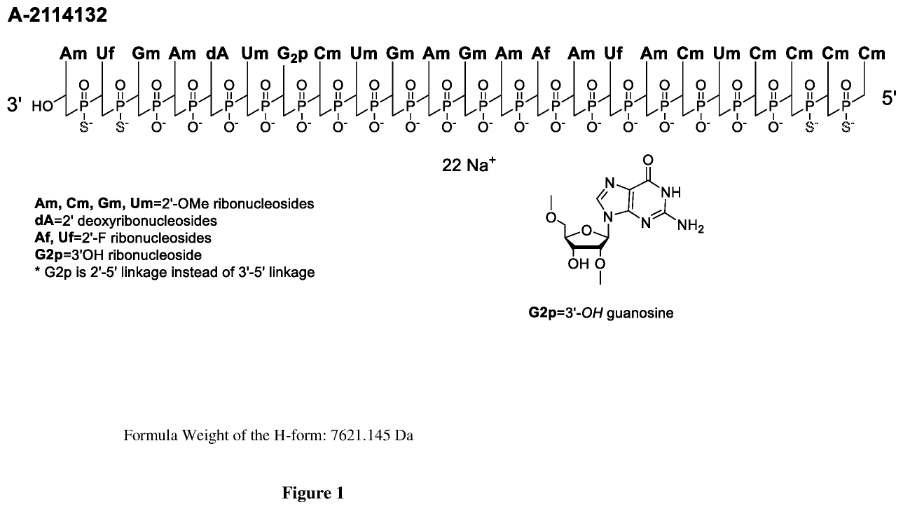

The structure of A-2114132 (SEQ ID NO: 1) is shown in FIG. 1. The accepted oligonucleotide nomenclatures for A-2114132 are:

5′ (2′-O-methyl) thioadenylyl-(3′-5′)-(2′-fluoro) thiouridylyl-(3′-5′)-(2′-O-methyl) guanylyl-(3′-5′)-(2′-O-methyl) adenylyl-(3′-5′)-(2′-deoxy) adenylyl-(3′-5′)-(2′-O-methyl) uridylyl-(2′-5′)-(3′-OH) guanylyl-(3′-5′)-(2′-O-methyl) cytidylyl-(3′-5′)-(2′-O-methyl) uridylyl-(3′-5′)-(2′-O-methyl) guanylyl-(3′-5′)-(2′-O-methyl) adenylyl-(3′-5′)-(2′-O-methyl) guanylyl-(3′-5′)-(2′-O-methyl) adenylyl-(3′-5′)-(2′-fluoro) adenylyl-(3′-5′)-(2′-O-methyl) adenylyl-(3′-5′)-(2′-fluoro) uridylyl-(3′-5′)-(2′-O-methyl) adenylyl-(3′-5′)-(2′-O-methyl) cytidylyl-(3′-5′)-(2′-O-methyl) uridylyl-(3′-5′)-(2′-O-methyl) cytidylyl-(3′-5′)-(2′-O-methyl) thiocytidylyl-(3′-5′)-(2′-O-methyl) thiocytidylyl-(3′-5′)-(2′-O-methyl) cytidine, 22 sodium salt 3′

The abbreviated sequence is: 5′ Am-ps-Uf-ps-Gm-Am-dA-Um-G2p-Cm-Um-Gm-Am-Gm-Am-Af-Am-Uf-Am-Cm-Um-Cm-Cm-ps-Cm-ps-Cm 3′

Uf = 2′-fluorouridine
Af = 2′-fluoroadenosine
Am = 2′-O-methyladenosine
Cm = 2′-O-methylcytidine
Gm = 2′-O-methylguanosine
Um = 2′-O-methyluridine
dA = 2′-deoxyadenosine
G2p = 3′-OH guanosine
‘-’ (hyphen) = 3′-5′ phosphodiester linkage
‘-ps-’ = 3′-5′ phosphorothioate linkage

### 1.2 Materials Used in the Synthesis

The starting materials used in the synthesis of A-2114132 included the 2′-OMe A loaded CPG as well as eight phosphoramidites. The CPG resin (Bz 2′-OMe A CPG) was purchased from LGC Biosearch Technologies. The protected phosphoramidites (2′-F U, 2′-F A, 2′-OMe A, 2′-OMe C, 2′-OMe G, 2′-OMe U, dA, and G2p) were purchased from Hongene Biotechnology USA, Inc. PADS, used for the thiolation step, was purchased from American International Chemical. TEAF was purchased from Millipore Sigma. The exocyclic amino groups were protected as acetyl (Ac 2′-OMe C phosphoramidites), benzoyl (Bz 2′-F A, Bz dA and Bz 2′-OMe A phosphoramidites), or isobutyryl (i-Bu dG, i-Bu G2p, and i-Bu 2′-OMe G phosphoramidites) amides, and the 5′-hydroxyl group was protected as the 4, 4′-dimethoxytrityl (DMT) ether in all cases. 3′-OH of the G2p was protected as 3′-OTIPs (

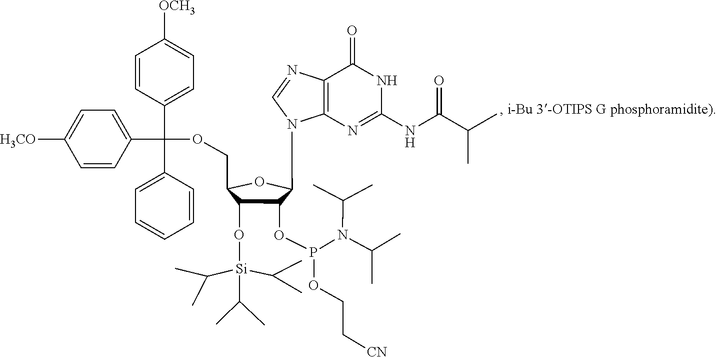

### 1.3 General Manufacture Process

The process for manufacturing the exemplified oligonucleotide containing a nucleotide having a 3′-hydroxyl group (3′-OH RNA modification) (e.g., A-2114132) included synthesis, cleavage and deprotection, 3′ desilylation, crude ultrafiltration, and purification.

In the solid phase synthesis of an oligonucleotide, each cycle consisted of 5′-deprotection by acid treatment, coupling, oxidation or thiolation, and capping. Each coupling reaction was carried out by activation of the appropriate amidite using 5-(ethylthio)-1H-tetrazole reagent followed by the coupling of the free 5′-hydroxyl group of a support-immobilized protected nucleoside or oligonucleotide. In the oxidation/thiolation step, the phosphodiester linkage or phosphorothioate linkage was created by converting ^{III}P (phosphite) to ^{V}P (phosphodiester) using I₂ solution in pyridine containing 10% water v/v, or converting ^{III}P (phosphite) to ^{V}P (phosphorothioate) using 0.2 M PADS in ACN/2,6-lutidine 1:1 v/v respectively. The unreacted free 5′-hydroxyl groups were then acetylated and thus “capped” to prevent elongation of unwanted “failure” sequences. After the appropriate number of cycles, the last 5′-protecting group DMT was removed by acid treatment followed by diethyl amine treatment to minimize the formation of cyanoethyl adducts.

After synthesis, the crude oligonucleotide of A-2114132 was cleaved from the solid support by aqueous ammonia/ethanol (3:1, v/v) treatment, with concomitant removal of nucleobase protecting groups.

The 3′-O-triisopropylsilyl (TIPS) group was deprotected by treatment with tetraethylammonium fluoride (TEAF) (C&D solution/DMSO, 1:1, v/v & 0.9M TEAF).

The crude oligonucleotide solution was then diluted before being subjected to ultrafiltration, followed by purification using strong anion exchange MPLC.

### 1.3.1 Solid Phase Synthesis

An exemplified oligonucleotide containing a nucleotide having a 3′-hydroxyl group (3′-OH RNA modification), A-2114132, was synthesized at 17.8 mmol scale using standard solid-phase oligonucleotide protocols on CPG loaded with DMT-protected 2′-OMe A. Each intermediate was assembled from the 3′ to the 5′ terminus by the addition of protected nucleoside phosphoramidites and an activator, except for G2p where the coupling direction was from 2′ to 5′ terminus. All the reactions took place on the derivatized support in a packed column. Briefly, phosphoramidites and activator (5-ethylthio-1H-tetrazole in ACN) ratio of 1:2 to 1:4 was used throughout the synthesis. The amidite required in each step was determined by the oligonucleotide sequence described above in Section 1.1 and listed in Section 1.2. Amidites were dissolved in acetonitrile, or in DMF (or DMF and a cosolvent). The amount of the protected nucleoside phosphoramidites was up to 3.0 equivalents. The activator was up to 12.0 equivalents. During coupling cycle, 5-44 minute recycle time was used to achieve desirable coupling. Following coupling, the column was put on an ABI for oxidation or sulfurization with a solution of iodine in pyridine in the presence of water or PADS in ACN/2,6-lutidine 1:1. The unreacted free 5′-hydroxyl groups were capped by using capping reagents (acetic anhydride/acetonitrile and N-methylimidazole/2,6-lutidine/acetonitrile).

### 1.3.2 Cleavage, Deprotection, Desilylation and Crude Ultrafiltration

Reiteration of the basic four-step cycle discussed above using the appropriate protected nucleoside phosphoramidites allowed the assembly of the entire protected sequence.

Several consequential deprotection steps, as shown in Scheme 1, were applied to result in a fully deprotected crude oligonucleotide in solution.

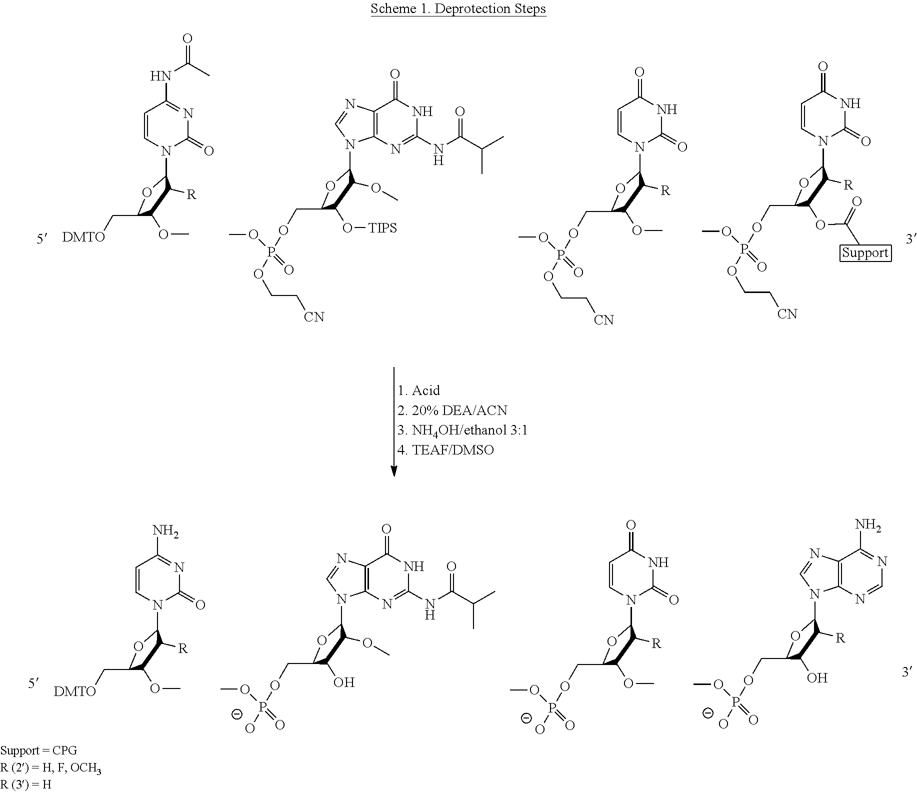

Step 1. DMT group protecting the 5′-hydroxyl of the oligonucleotide chain was removed from the solid-supported oligonucleotide through an acid treatment (on column treatment, integrated into automated cycle).

Step 2. Cyanoethyl protecting groups were removed using diethyl amine treatment (20% DEA in ACN) while the oligonucleotide was still attached to the support (on column treatment, integrated into automated cycle).

Step 3. The crude oligonucleotide was cleaved from the solid support by shaking the solid support attached oligo with 30% ammonia/ethanol (3:1, v/v) at 35° C. for 16-20 hours in Pyrex bottles. Removal of the nucleobase protecting groups occurred concomitantly with the cleavage.

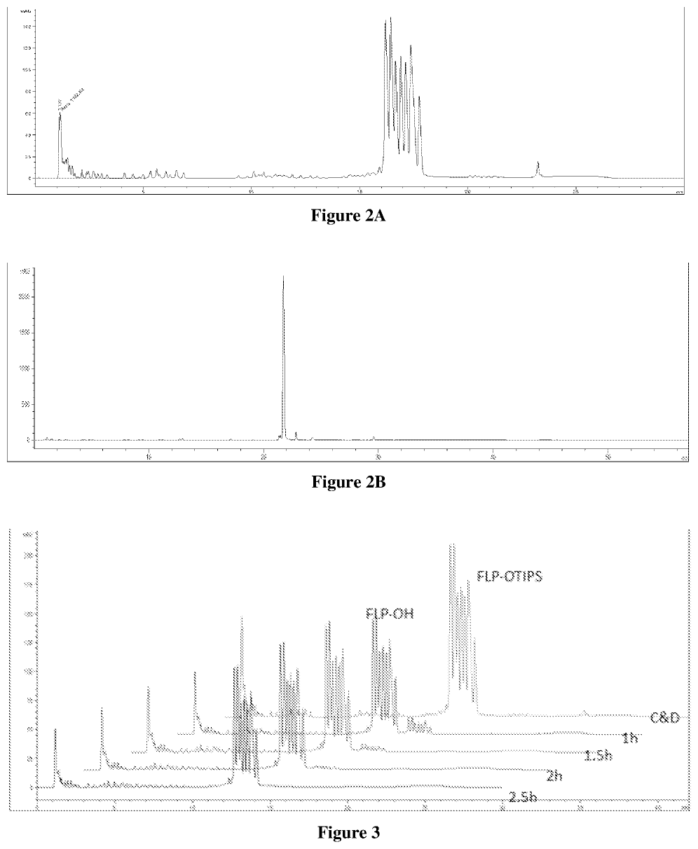

At the end of the treatment, the 3′-OH group of the G2p unit was still protected with triisopropylsilyl (TIPS) group, as indicated from the HPLC results shown in FIGS. 2A and 2B. The reaction mixture was filtered, and the solid support cake was washed with DMSO. Foam was observed during filtration which was disrupted when vacuum was released. The filtrate (the oligonucleotide solution) and DMSO washes were combined in a jacketed reactor for the desilylation step.

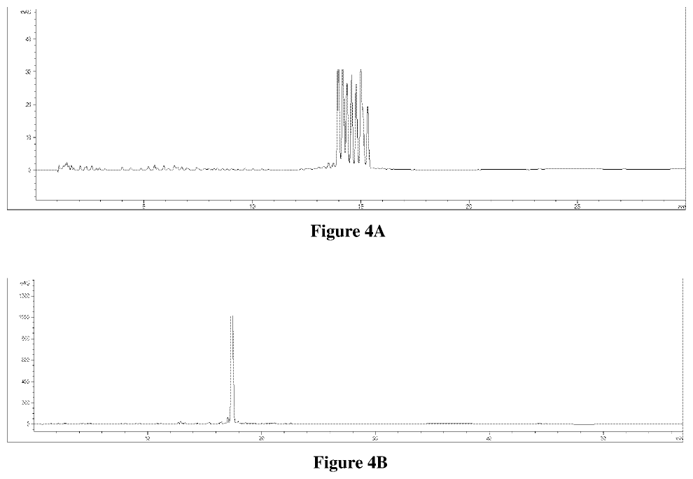

Step 4. The O-TIPS protective group removal from the 3′-OH of G2p unit (desilylation) was performed using TEAF in aqueous DMSO solution (126.5 mL/mmol) at 60° C. TEAFxH₂O (38 g/mmol) was added as a solid in portions over 10 minutes. The addition of TEAF was not noticeably exothermic; thus, the order of addition could be changed, i.e., TEAF could be loaded to the reactor prior to the loading of the oligonucleotide solution and/or DMSO. The mixture was heated to 60° C. while stirring. Some amount of gas (mainly ammonia) was evolved, causing substantial foaming during the heating. The intensity of foaming was controlled by adjusting the heating rate and stirring rate. Clear solution was formed in 10 minutes. The mixture was stirred at 60° C. for 2 hours to remove the TIPS protective group, monitored by AX-HPLC, as shown in FIG. 3. The mixture was then cooled, and diluted with water. The FLP purity (by IEX) was 75.0% (by IEX) and 76.9% FLP (by IPRP). The crude yield was 120 (OD//μmol).

The crude mixture was then desalted using a standard ultrafiltration process with a regenerated cellulose as the membrane (5 kD MWCO). The crude ultrafiltration step was performed to remove basic ammonia solution and other small molecules from cleavage, deprotection, and desilylation steps, thereby minimizing the potential side reactions of 2′-F nucleosides occurring during subsequent MPLC purification at elevated temperature.

The desalted crude material was subjected to HPLC analysis and the results are shown in FIGS. 4A and 4B. The FLP recovery was 100%, with a purity of 80.8% by AX-HPLC, and a purity of 80.5 by IPRP-HPLC.

### Example 2. Manufacture of an Exemplified 3′-RNA Containing Oligonucleotide

The manufacture of an exemplified oligonucleotide containing a nucleotide having a 3′-hydroxyl group (3′-OH RNA modification) (A-2403673) is illustrated in this example by solid phase synthesis using 3′-O-(2-cyanoethyl) phosphoramidite chemistry, except for C2p which uses 2′-O-(2-cyanoethyl) phosphoramidite chemistry followed by chromatographic purification. The synthesis is performed on 2′-OMe G loaded controlled pore glass (CPG) support.

### 2.1 Structure and Sequence of A-2403673

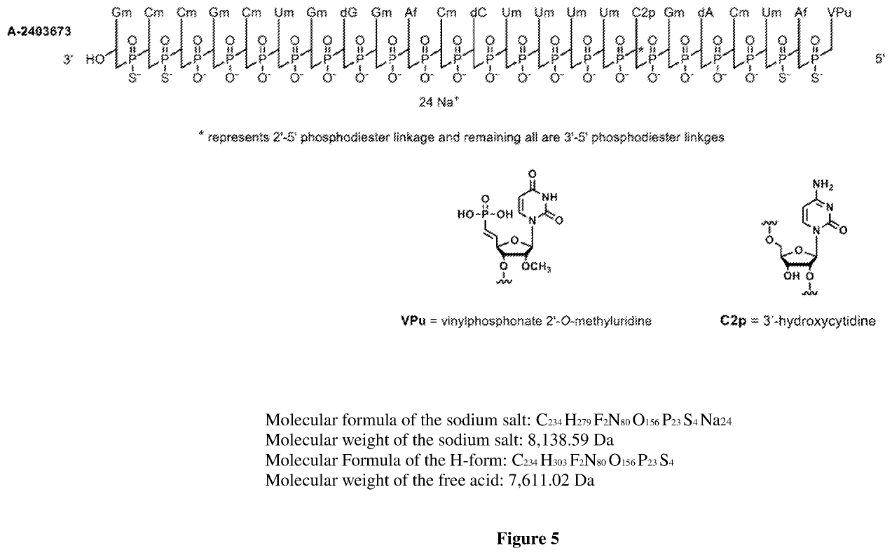

The structure of A-2403673 (SEQ ID NO:2) is shown in FIG. 5. The accepted oligonucleotide nomenclatures for A-2403673 are:

5′ (Vinylphosphonate-2′-O-methyl)thiouridylyl-(3′-5′)-(2′-fluoro)thioadenylyl-(3′-5′)-(2′-Omethyl)uridylyl-(3′-5′)-(2′-O-methyl)cytidylyl-(3′-5′)-(2′-deoxy)adenylyl-(3′-5′)-(2′-Omethyl)guanylyl-(3′-5′)-(3′-hydroxyl)cytidylyl-(2′-5′)-(2′-O-methyl)uridylyl-(3′-5′)-(2′-Omethyl)uridylyl-(3′-5′)-(2′-O-methyl)uridylyl-(3′-5′)-(2′-O-methyl)uridylyl-(3′-5′)-(2′-deoxy)cytidylyl-(3′-5′)-(2′-O-methyl)cytidylyl-(3′-5′)-(2′-fluoro)adenylyl-(3′-5′)-(2′-O methyl)guanylyl-(3′-5′)-(2′-deoxy)guanylyl-(3′-5′)-(2′-O-methyl)guanylyl-(3′-5′)-(2′-Omethyl)uridylyl-(3′-5′)-(2′-O-methyl)cytidylyl-(3′-5′)-(2′-O-methyl)guanylyl-(3′-5′)-(2′-Omethyl)thiocytidylyl-(3′-5′)-(2′-O-methyl)thiocytidylyl-(3′-5′)-(2′-O-methyl)guanidine, 24 sodium salt 3′

The abbreviated sequence is: 5′ VPu-ps-Af-ps-Um-Cm-dA-Gm-C2p-Um-Um-Um-Um-dC-Cm-Af-Gm-dG-Gm-Um-Cm-Gm-Cm-ps-Cm-ps-Gm 3′

Uf = 2′-fluorouridine
Af = 2′-fluoroadenosine
Cf = 2′-fluorocytidine
Gf = 2′-fluoroquonosine
Am = 2′-O-methyladenosine
Cm = 2′-O-methylcytidine
Gm = 2′-O-methylguanosine
Um = 2′-O-methyluridine
dA = 2′-deoxyadenosine
dC = 2′-deoxycytidine
dG = 2′-deoxyguanidine
C2p = 3′-OH-cytidine
VPu = vinylphosphonate 2′-O-methyluridine
‘-’ (hyphen) = 3′-5′ phosphodiester linkage
‘-ps-’ = 3′-5′ phosphorothioate linkage

### 2.2 Materials for the Synthesis

The starting materials used in the synthesis of A-2403673 include the 2′-OMe G loaded CPG (e.g., CPG resin (Bz 2′-OMe G CPG)) as well as nine phosphoramidites (2′-F A, 2′-OMe C, 2′-OMe G, 2′-OMe U, dA, dC, dG, VP 2′-OMe U, C2p). The exocyclic amino groups can be protected as acetyl (Ac 2′-OMe C, Ac dC, and Ac C2p phosphoramidites), benzoyl (Bz 2′-F A, Bz dA, and Bz 2′-OMe U phosphoramidites), or isobutyryl (i-Bu dG, and i-Bu 2′-OMe G phosphoramidites) amides, and the 5′-hydroxyl group are protected as the 4, 4′-dimethoxytrityl (DMT) ether in all cases. For VP 2′-OMe U VP 2′-OMe U, 5′-hydroxyl group is protected as

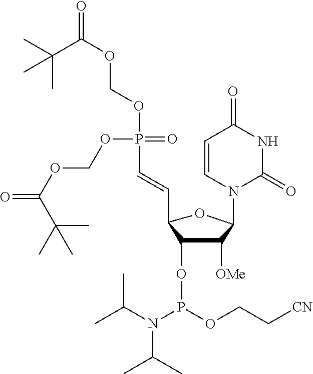

(VP 2′-OMe U phosphoramidite; 3′-O-[(N,N-diisopropylamino)-2-cyanoethoxyphosphinyl)-((E)-(4′-vinylphosphoryl)bis(oxy)bis(methylene)bis(2,2-dimethylpropanoate))-2′-O-methyluridine; MW 762.73). 3′-OH of the C2p is protected as 3′-OTIPs

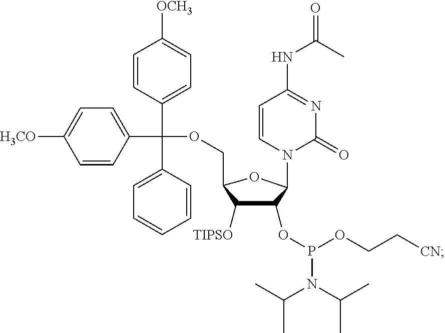

Ac 3′-OTIPS C phosphoramidite; 2′-O-[(N,N-diisopropylamino)-2-cyanoethoxyphosphinyl]-5′-O-(4,4′-dimethoxytrityl)-3′-O-triisopropylsilyl-N-acetylcytidine; MW 944.18).

### 2.3 General Manufacture Process

The process for manufacturing the exemplified oligonucleotide containing a nucleotide having a 3′-hydroxyl group (3′-OH RNA modification), A-2403673, is similar to the procedures described in Example 1, and includes synthesis, cleavage and deprotection, 3′ desilylation, crude ultrafiltration, and purification.

In the solid phase synthesis, each cycle consists of 5′-deprotection by acid treatment, coupling, oxidation or thiolation, and capping. Each coupling reaction is carried out by activation of the appropriate amidite using 5-(ethylthio)-1H-tetrazole reagent followed by the coupling of the free 5′-hydroxyl group of a support-immobilized protected nucleoside or oligonucleotide. In the oxidation/thiolation step, the phosphodiester linkage or phosphorothioate linkage is created by converting ^{III}P (phosphite) to ^{V}P (phosphodiester) (e.g., using I₂ solution in pyridine (e.g., a solution of iodine in pyridine containing 10% water v/v), or converting ^{III}P (phosphite) to ^{V}P (phosphorothioate) (e.g., using PADS in ACN/2,6-lutidine, for instance, 0.2 M PADS in ACN/2,6-lutidine 1:1 v/v) respectively. The unreacted free 5′-hydroxyl groups are then acetylated and thus “capped” to prevent elongation of unwanted “failure” sequences.

After synthesis, the crude oligonucleotide of A-2403673 is cleaved from the solid support by aqueous amine (or ammonia or methylamine)/ethanol (e.g., 3:1, v/v) treatment optionally in the presence of up to 5% DMSO by volume, with concomitant removal of nucleobase and phosphate protecting groups protecting groups.

The 3′-O-triisopropylsilyl (TIPS) group is deprotected by treatment with tetraethylammonium fluoride (TEAF) (e.g., C&D solution/DMSO, 1:1, v/v & 0.9M TEAF).

The crude oligonucleotide solution is then diluted before being subjected to ultrafiltration, followed by purification (e.g., using strong anion exchange MPLC).

### 2.3.1 Solid Phase Synthesis

A-2114132 is synthesized using standard solid-phase oligonucleotide protocols on CPG loaded with DMT-protected 2′-OMe G. Each intermediate is assembled from the 3′ to the 5′ terminus by the addition of protected nucleoside phosphoramidites and an activator, except for C2p where the coupling direction is from 2′ to 5′ terminus. All the reactions take place on the derivatized support in a packed column. Phosphoramidites and activator (e.g., 5-ethylthio-1H-tetrazole in ACN) in a ratio of 1:2 to 1:4 may be used throughout the synthesis. The amidite required in each step is determined by the oligonucleotide sequence described above in Section 2.1 and listed in Section 2.2. Amidites may be dissolved in acetonitrile, or in DMF (or DMF and a cosolvent). The amount of the protected nucleoside phosphoramidites may be up to 3.0 equivalents. The activator may be up to 12.0 equivalents. During coupling cycle, 5-44 minute recycle time may be used to achieve desirable coupling. Following coupling, the column can be put on an ABI for oxidation or sulfurization with a solution of iodine in pyridine in the presence of water or PADS in ACN/2,6-lutidine (e.g., 1:1). The unreacted free 5′-hydroxyl groups are capped by using capping reagents (acetic anhydride/acetonitrile and N-methylimidazole/2,6-lutidine/acetonitrile).

### 2.3.2 Cleavage, Deprotection, Desilylation and Crude Ultrafiltration

Reiteration of the basic four-step cycle discussed above using the appropriate protected nucleoside phosphoramidites allows the assembly of the entire protected sequence.

Several consequential deprotection steps, as shown in Scheme 1, can be applied to result in a fully deprotected crude oligonucleotide in solution.

Step 1. DMT group protecting the 5′-hydroxyl of the oligonucleotide chain can be removed from the solid-supported oligonucleotide through an acid treatment (on column treatment, integrated into automated cycle).

Step 2. Cyanoethyl protecting groups can be removed using diethyl amine treatment (e.g., 20% DEA in ACN) while the oligonucleotide is still attached to the support (on column treatment, integrated into automated cycle).

Step 3. The crude oligonucleotide can be cleaved from the solid support by shaking the solid support attached oligo with ammonia/ethanol (e.g., 30% ammonia/ethanol (3:1, v/v)) at about 33 to about 37° C. for 12-22 (e.g., 16-20 hours) in Pyrex bottles. Removal of the nucleobase protecting groups may occur concomitantly with the cleavage.

At the end of the treatment, the 3′-OH group of the C2p unit may be still protected with triisopropylsilyl (TIPS) group. The reaction mixture can be filtered, and the solid support cake is washed with DMSO. The filtrate (the oligonucleotide solution) and DMSO washes can be combined in a jacketed reactor for the desilylation step.

Step 4. The O-TIPS protective group removal from the 3′-OH of C2p unit (desilylation) can be performed using TEAF in aqueous DMSO solution (e.g., about 0.9 M TEAF in DMSO) at a temperature ranging from about 60° C. to about 65° C. (e.g., about 60° C.). TEAFxH₂O may be added as a solid in portions over a period of time (e.g., 10 minutes). The mixture is stirred at about 60° C. to about 65° C. (e.g., about 60° C.) for several hours (e.g., 2 hours) to remove the TIPS protective group, monitored by AX-HPLC. The mixture is then cooled, and diluted with water.

The crude mixture is then desalted using a standard ultrafiltration process (e.g., with a regenerated cellulose as the membrane (e.g., 5 kD MWCO). The crude ultrafiltration step is performed to remove basic ammonia solution and other small molecules from cleavage, deprotection, and desilylation steps, thereby minimizing the potential side reactions of 2′-F nucleosides occurring during subsequent MPLC purification at elevated temperature.

We claim:

1. A 3′-hydroxyl protected oligonucleotide intermediate having the structure of formula (I):
A-[C]ₙ-E (I), or a salt thereof,
wherein:
A is 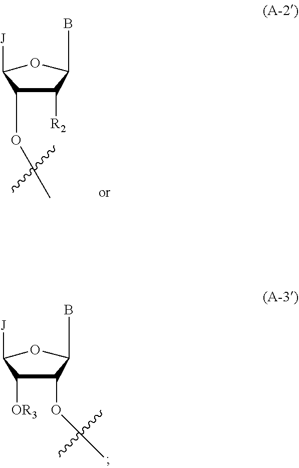
C is 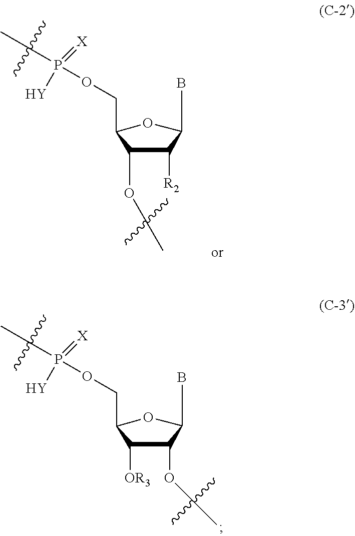
E is 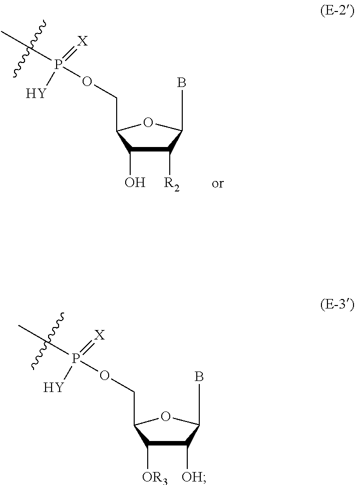
provided that the oligonucleotide intermediate contains at least one (A-3′), (C-3′), or (E-3′);
n is 1-50;
B is a modified or unmodified nucleobase;
J is 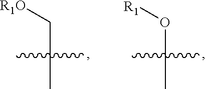
or a phosphorous-containing group or moiety;
R₁ is H, a hydroxyl protecting group, or a phosphorous-containing group or moiety;
R₂ is H, halo, OR₅, or NR₆R₇;
R₃ is —(CH₂)ₘSi(R₄)₃;
each R₄ is independently optionally substituted alkyl, aryl, aralkyl, alkaryl, cycloalkyl, alkenyl, cycloalkenyl, alkynyl or cycloalkynyl;
m is 0, 1, 2, or 3;
R₅, R₆, and R₇ are each independently H or optionally substituted alkyl, alkoxyalkyl, aryl, aralkyl, alkaryl, cycloalkyl, alkenyl, cycloalkenyl, alkynyl or cycloalkynyl; or R₆ and R₇ are linked to form a heterocyclyl; and
X and Y are each independently O or S.

2. The oligonucleotide intermediate of claim 1, wherein n is 16 to 23.

3. The oligonucleotide intermediate of claim 1, wherein A is A-3′.

4. The oligonucleotide intermediate of claim 1, wherein at least one C is C-3′.

5. The oligonucleotide intermediate of claim 1, wherein E is E-3′.

6. The oligonucleotide intermediate of claim 1, wherein m is 0 or 1, and each R₄ is isopropyl.

7. The oligonucleotide intermediate of claim 1, wherein R₂ is H, F, OCH₃, or OCH₂CH₂OCH₃.

8. A method of deprotecting a 3′-hydroxyl of an oligonucleotide having the 3′-hydroxyl protected by a silyl protecting group, comprising:
contacting the oligonucleotide with a mixture comprising DMSO and a deprotecting reagent having a formula of (R)₄N⁺F⁻, wherein each R is independently alkyl, aryl, heteroaryl, aralkyl, or heteroaralkyl, under conditions sufficient for the deprotecting reagent to remove the silyl protecting group.

9. The method of claim 8, wherein the silyl protecting group is trialkylsilyl.

10. The method of claim 8, wherein the silyl protecting group is triisopropylsilyl (TIPS) or tert-butyldimethylsilyl (TBDMS).

11. The method of claim 8, wherein the deprotecting reagent is tetraethylammonium fluoride (TEAF) or tetra-n-butylammonium fluoride (TBAF).

12. The method of claim 11, wherein the deprotecting reagent is tetraethylammonium fluoride hydrate (TEAF⋅xH₂O).

13. The method of claim 11, wherein the mixture is 0.9 M TEAF in aqueous DMSO solution.

14. The method of claim 11, wherein the contacting step is carried out at 60° C. or higher.

15. The method of claim 11, wherein the contacting step is carried out for 30 minutes or longer.

16. The method of claim 15, wherein the contacting step is carried out for 2 hours or longer.

17. The method of claim 8, further comprising, prior to removing the silyl protecting group:
contacting a nucleobase-protected oligonucleotide having a 3′-hydroxyl protected by a silyl protecting group bound to a solid support with a base under conditions suitable to cleave the oligonucleotide from the solid support and to remove amino protecting groups from the nucleobase-protected oligonucleotide, to provide the oligonucleotide having the 3′-hydroxyl protected by a silyl protecting group.

18. The method of claim 17, wherein the base is ammonia in a solvent.

19. The method of claim 18, wherein the amino protecting groups are exocyclic amino (nucleobase) protecting groups, and the base is a mixture of 30% ammonia in a solvent at a v/v ratio of no more than 3:1.

20. The method of claim 17, further comprising, prior to the base treatment step, contacting a fully-protected oligonucleotide having a 3′-hydroxyl protected by a silyl protecting group bound to a solid support with a base under conditions suitable to remove a phosphate protecting group of the oligonucleotide, to provide the nucleobase-protected oligonucleotide having a 3′-hydroxyl protected by a silyl protecting group.

21. The method of claim 20, wherein the base is a weak base.

22. The method of claim 21, wherein the weak base is diethyl amine (DEA).

23. The method of claim 22, wherein the phosphate protecting group is cyanoethyl, and the weak base is 20% DEA in acetonitrile.

24. The method of claim 17, further comprising the step of purifying the oligonucleotide via a chromatographic purification.
